(19) United States
(12) Patent Application Publication
(10) Pub. No.: US 2025/0255792 A1
(43) Pub. Date: Aug. 14, 2025

(54) Methods of Using a Small Molecule Chemical Compound to Reduce the Appearance of Rhytids and Wrinkles

(71) Applicant: BirchBioMed Inc., King City (CA)

(72) Inventors: Mark S. Miller, King City (CA); Jonathan W. Bourne, Fairport, NY (US)

(21) Appl. No.: 19/111,514

(22) PCT Filed: Sep. 14, 2023
(86) PCT No.: PCT/CA2023/051220

§ 371 Date: Mar. 13, 2025

Related U.S. Application Data
(60) Provisional application No. 63/406,456, filed on Sep. 14, 2022.

(51) Int. Cl. A61K 8/44; A61K 8/98; A61Q 19/08

(52) U.S. Cl. CPC A61K 8/445; A61K 8/983; A61Q 19/08; A61K 2800/91

(57) ABSTRACT

This invention relates to the field of dermatology and cosmetology and describes methods of using small molecule chemical compounds and formulations thereof to reduce the appearance of rhytids and wrinkles. These methods and formulations are contemplated for cosmetic uses of reducing the appearance of rhytids or wrinkles, or for potential therapeutic uses of reducing, reversing, or treating rhytids or wrinkles. Also contemplated are use of these compounds to reduce the appearance of rhytids or wrinkles or for potential therapeutic uses of reducing, reversing, or treating rhytids or wrinkles.

### FIELD OF THE INVENTION

The present invention relates to methods of reducing the appearance of rhytids or wrinkles on a person by applying or administering a small molecule chemical compound. Reducing the appearance of rhytids or wrinkles is desirable for aesthetic purposes and cosmetic purposes, and methods and compositions to reduce the appearance of these would be commercially desirable to the cosmetic and pharmaceutical industries as well as to manufacturers of chemicals.

### BACKGROUND OF THE INVENTION

The elderly are making up an increase fraction of the global population, with individuals older than 65 projected to be almost 40% of the US population by 2030 (Venkatesh, 2019). According to Chaudhary et al. (2021), by 2050 more than 2 billion of the global population will be >60 years of age. With this increasing aging of the global population the worldwide anti-aging market size (including products, services, and devices) is projected by some to exceed a $270 billion USD by 2024, and competition is increasing among cosmetic brands to address this growing anti-aging market (Ferreira, 2020).

Hallmarks of skin aging include histological changes such as thinning of the skin due to atrophy of the epidermal cell layers and reduction in the components of the extracellular matrix (ECM) in the dermal layer (for example collagen decreases and the collagen becomes fragmented) where the reduction in the ECM and collagen fragmentation is attributable to increased secretion of matrix metalloproteinases by skin fibroblasts with aging (Salminen, 2022). According to Saminen et al., changes in the amount of ECM components “result in many of the clinical features encountered in the aged skin, such as wrinkles and loss of skin elasticity.” (2022, pg. 818)

Venkatesh et al., similarly states that chronological aging is marked by clinical changes such as skin thinning and wrinkles (2019). The skin dermis exhibits decreased collagen synthesis and degraded elastic fibers, and Venkatesh et al. states “[d]egraded elastic fibers and reduced collagen synthesis are responsible for reduced skin elasticity, or skin ptosis, and these alterations contribute to rhytids, or wrinkles.” (2019, pg. 352).

According to Kim et al., MMPs are a major driver in photo-induced aging of skin although he reports that MMP2 is the major degrading enzyme and that photoaging is induced in large part by aryl hydrocarbon receptor (AhR) activation and they then showed that aryl hydrocarbon receptor antagonists (AhR inhibition) using vitamin B12 and folic acid ameliorated UVB-induced wrinkle formation (2022). Those authors further reported that Kyat2, which is an enzyme involved in the endogenous production of kynurenic acid and noting that kynurenic acid is a major endogenous ligand for AhR stimulation (kynurenic acid is an AhR agonist or AhR activator), expression positively correlated with MMP2 and MMP11 mRNA expression (2022). These data therefore indicates that kynurenic acid, which was previously reported to upregulate MMP1 and MMP3 (Poormasjedi-Meibod, 2014), also increases MMP2 expression and generally contributes to ECM degradation in skin.

In light of high global prevalence of rhytids and wrinkles, new compositions and methods to reduce, reverse, or treat rhytids or wrinkles or to reduce the appearance of rhytids or wrinkles continue to be of commercial and industrial interest for both medical and cosmetic purposes.

### SUMMARY OF THE INVENTION

The present invention relates to formulations and methods of reducing or decreasing the appearance of rhytids or wrinkles on a person by applying or administering a small molecule chemical compound. Reducing the appearance of rhytids and wrinkles, or treatments to reduce rhytids and wrinkles are desirable for cosmetic, aesthetic, and medical reasons.

In one embodiment, the present invention contemplates a method to reduce the appearance of rhytids or wrinkles comprising of administering or applying a small molecule compound wherein the compound is selected from the group consisting of DL-kynurenine, L-kynurenine, D-kynurenine, 3-hydroxy-DL-kynurenine, 3-hydroxy-L-kynurenine, 3-hydroxy-D-kynurenine, 5-hydroxy-DL-kynurenine, 5-hydroxy-L-kynurenine, 5-hydroxy-D-kynurenine, N′-formyl-kynurenine, N-Acetyl-3-OH-kynurenine, 4-Chloro-DL-kynurenine, Xanthurenic Acid, and Kynurenic Acid.

In one embodiment, the present invention contemplates a method to treat the appearance of rhytids or wrinkles comprising of administering or applying a small molecule compound wherein the compound is selected from the group consisting of DL-kynurenine, L-kynurenine, D-kynurenine, 3-hydroxy-DL-kynurenine, 3-hydroxy-L-kynurenine, 3-hydroxy-D-kynurenine, 5-hydroxy-DL-kynurenine, 5-hydroxy-L-kynurenine, 5-hydroxy-D-kynurenine, N′-formyl-kynurenine, N-Acetyl-3-OH-kynurenine, 4-Chloro-DL-kynurenine, Xanthurenic Acid, and Kynurenic Acid.

In one embodiment, the present invention contemplates a method to treat rhytids or wrinkles comprising of administering or applying a small molecule compound wherein the compound is selected from the group consisting of DL-kynurenine, L-kynurenine, D-kynurenine, 3-hydroxy-DL-kynurenine, 3-hydroxy-L-kynurenine, 3-hydroxy-D-kynurenine, 5-hydroxy-DL-kynurenine, 5-hydroxy-L-kynurenine, 5-hydroxy-D-kynurenine, N′-formyl-kynurenine, N-Acetyl-3-OH-kynurenine, 4-Chloro-DL-kynurenine, Xanthurenic Acid, and Kynurenic Acid.

In one embodiment, the present invention contemplates a method to reverse rhytids or wrinkles comprising of administering or applying a small molecule compound wherein the compound is selected from the group consisting of DL-kynurenine, L-kynurenine, D-kynurenine, 3-hydroxy-DL-kynurenine, 3-hydroxy-L-kynurenine, 3-hydroxy-D-kynurenine, 5-hydroxy-DL-kynurenine, 5-hydroxy-L-kynurenine, 5-hydroxy-D-kynurenine, N′-formyl-kynurenine, N-Acetyl-3-OH-kynurenine, 4-Chloro-DL-kynurenine, Xanthurenic Acid, and Kynurenic Acid.

In one embodiment, the present invention contemplates a method to prevent rhytids or wrinkles comprising of administering or applying a small molecule compound wherein the compound is selected from the group consisting of DL-kynurenine, L-kynurenine, D-kynurenine, 3-hydroxy-DL-kynurenine, 3-hydroxy-L-kynurenine, 3-hydroxy-D-kynurenine, 5-hydroxy-DL-kynurenine, 5-hydroxy-L-kynurenine, 5-hydroxy-D-kynurenine, N′-formyl-kynurenine, N-Acetyl-3-OH-kynurenine, 4-Chloro-DL-kynurenine, Xanthurenic Acid, and Kynurenic Acid.

In one embodiment, the present invention contemplates a method to conceal rhytids or wrinkles comprising of administering or applying a small molecule compound wherein the compound is selected from the group consisting of DL-kynurenine, L-kynurenine, D-kynurenine, 3-hydroxy-DL-kynurenine, 3-hydroxy-L-kynurenine, 3-hydroxy-D-kynurenine, 5-hydroxy-DL-kynurenine, 5-hydroxy-L-kynurenine, 5-hydroxy-D-kynurenine, N′-formyl-kynurenine, N-Acetyl-3-OH-kynurenine, 4-Chloro-DL-kynurenine, Xanthurenic Acid, and Kynurenic Acid.

In one embodiment, the present invention contemplates a method to treat, reverse, conceal, or reduce the appearance of rhytids comprising of administering or applying a small molecule compound wherein the compound is selected from the group consisting of DL-kynurenine, L-kynurenine, D-kynurenine, 3-hydroxy-DL-kynurenine, 3-hydroxy-L-kynurenine, 3-hydroxy-D-kynurenine, 5-hydroxy-DL-kynurenine, 5-hydroxy-L-kynurenine, 5-hydroxy-D-kynurenine, N′-formyl-kynurenine, N-Acetyl-3-OH-kynurenine, 4-Chloro-DL-kynurenine, Xanthurenic Acid, and Kynurenic Acid.

In one embodiment, the present invention contemplates a method to treat, reverse, conceal, or reduce the appearance of wrinkles comprising of administering or applying a small molecule compound wherein the compound is selected from the group consisting of DL-kynurenine, L-kynurenine, D-kynurenine, 3-hydroxy-DL-kynurenine, 3-hydroxy-L-kynurenine, 3-hydroxy-D-kynurenine, 5-hydroxy-DL-kynurenine, 5-hydroxy-L-kynurenine, 5-hydroxy-D-kynurenine, N′-formyl-kynurenine, N-Acetyl-3-OH-kynurenine, 4-Chloro-DL-kynurenine, Xanthurenic Acid, and Kynurenic Acid.

In one embodiment, the present invention contemplates a compound selected from the group consisting of DL-kynurenine, L-kynurenine, D-kynurenine, 3-hydroxy-DL-kynurenine, 3-hydroxy-L-kynurenine, 3-hydroxy-D-kynurenine, 5-hydroxy-DL-kynurenine, 5-hydroxy-L-kynurenine, 5-hydroxy-D-kynurenine, N′-formyl-kynurenine, N-Acetyl-3-OH-kynurenine, 4-Chloro-DL-kynurenine, Xanthurenic Acid, and Kynurenic Acid, for use in the treatment of rhytids or wrinkles. In one embodiment, the compound is kynurenic acid for use in the treatment of rhytids or wrinkles. In one embodiment, kynurenic acid is a component of a composition formulated for topical administration for use in the treatment of rhytids or wrinkles. In one embodiment, the compound is kynurenine for use in the treatment of rhytids or wrinkles.

In one embodiment, the compound is a component of a composition formulated for injection and is administered by injection. In one embodiment the injection is administered as a subcutaneous, intradermal, or an intramuscular injection. In another embodiment, the compound is a component of a composition formulated for platelet-rich plasma (PRP) therapy or platelet-rich fibrin (PRF) therapy. In yet another embodiment, the compound is a component of a composition formulated for microneedling with PRP or PRF. In a further embodiment, the compound is administered or applied in combination with platelet-rich plasma (PRP) therapy or platelet-rich fibrin (PRF) therapy or microneedling.

In another embodiment, the compound is a component of a composition formulated for platelet-rich plasma (PRP) therapy or platelet-rich fibrin (PRF) therapy. In yet another embodiment, the compound is a component of a composition formulated for microneedling with PRP or PRF. In a further embodiment, the compound is administered or applied in combination with platelet-rich plasma (PRP) therapy or platelet-rich fibrin (PRF) therapy. The PRP or the PRF may be co-administered with the compound. The co-administration may be simultaneous. The co-administration may be consecutive. The PRP or PRF may be administered before the compound. The compound may be administered before the PRP or PRF.

In another embodiment, an antigen presenting cell (APC) is presented to a mammal and the compound is administered to the mammal. The APC may be allogenic. The APC may be syngenic. The APC may be a non-professional APC. The APC may be a professional APC. The APC and the compound may be co-administered. The co-administration may be simultaneous. The co-administration may be consecutive. The APC may be administered before the compound. The compound may be administered before the APC. The APC may be administered intraperitoneally. The compound may be administered orally. The APC may be selected from one or more of the following: fibroblasts, thymic epithelial cells, thyroid epithelial cells, glial cells, beta-cells, and endothelial cells. The APCs may be fibroblast cells. The mammal may be a human. The mammal may be a human with rhytids and/or wrinkles. The mammal may be a human with rhytids and/or wrinkles, the APC may be a fibroblast. The method may further include periodic compound administration following the initial administration of APC and the compound. The compound may be selected from one of more of DL-kynurenine, L-kynurenine, D-kynurenine, and 3-hydroxy-DL-kynurenine. The kynurenine may be selected from one of more of DL-kynurenine, L-kynurenine, D-kynurenine, 3-hydroxy-DL-kynurenine, 3-hydroxy-L-kynurenine, 3-hydroxy-D-kynurenine, 5-hydroxy-DL-kynurenine, 5-hydroxy-L-kynurenine, 5-hydroxy-D-kynurenine, N′-formyl-kynurenine, N-Acetyl-3-OH-kynurenine, 4-Chloro-DL-kynurenine, Kynurenine, butyl ester, and Kynurenic Acid. The kynurenine may be an isomer of kynurenine or an analog thereof.

In one embodiment, the compound is a component of a composition formulated for oral delivery. In one embodiment the compound is an ingredient in a product intended for oral consumption.

In one embodiment, the compound is a component of a composition formulated for topical delivery. In one embodiment, the compound is a component of a composition formulated for topical application. In one embodiment the compound is an ingredient in a lotion, cream, gel, solution, or suspension intended for topical use. In one embodiment, the compound is 0.05 to 10% by weight of the lotion, cream, gel, solution, or suspension. In one embodiment, the compound is 0.05 to 6% by weight of the lotion, cream, gel, solution, or suspension. In one embodiment, the compound is 0.05 to 3% by weight of the lotion, cream, gel, solution, or suspension. In one embodiment, the compound is 0.1 to 1% by weight of the lotion, cream, gel, solution, or suspension. In one embodiment, the compound is 0.1 to 0.5% by weight of the lotion, cream, gel, solution, or suspension. In one embodiment, the compound is 0.25 to 0.5% by weight of the lotion, cream, gel, solution, or suspension. In one embodiment, the compound is kynurenic acid. In one embodiment the compound is kynurenine.

In one embodiment, kynurenic acid is 0.1 to 1% by weight of the lotion, cream, gel, solution, or suspension. In one embodiment, kynurenic acid is 0.1 to 0.5% by weight of the lotion, cream, gel, solution, or suspension. In one embodiment, kynurenic acid is 0.5% by weight of the lotion, cream, gel, solution, or suspension. In one embodiment, kynurenic acid is 0.25% by weight of the lotion, cream, gel, solution, or suspension. In one embodiment, kynurenic acid is 0.1% by weight of the lotion, cream, gel, solution, or suspension.

In one embodiment, kynurenine is 0.1 to 1% by weight of the lotion, cream, gel, solution, or suspension. In one embodiment, kynurenine is 0.1 to 0.5% by weight of the lotion, cream, gel, solution, or suspension. In one embodiment, kynurenine is 0.5% by weight of the lotion, cream, gel, solution, or suspension. In one embodiment, kynurenine is 0.25% by weight of the lotion, cream, gel, solution, or suspension. In one embodiment, kynurenine is 0.1% by weight of the lotion, cream, gel, solution, or suspension.

In some embodiments, the application, administration, or use of the compound is contemplated as once, twice, or thrice daily. In some embodiments, the application, administration, or use of the compound is contemplated as once, twice, or thrice daily, wherein the compound is kynurenine or kynurenic acid. In some embodiments, the application, administration, or use is of a lotion, cream, gel, solution, or suspension that contains kynurenine or kynurenic acid at between 0.1 to 3% by weight.

### BRIEF DESCRIPTION OF THE DRAWINGS

FIG. 1A shows the relative expression of collagen 1 (A) protein in dermal fibroblasts normalized to beta-actin after being treated with kynurenine and kynurenic acid (reproduced from Poormasjedi-Meibod et al. (2014), FIG. 1).

FIG. 1B shows the relative expression of fibronectin (B) protein in dermal fibroblasts normalized to beta-actin after being treated with kynurenine and kynurenic acid (reproduced from Poormasjedi-Meibod et al. (2014), FIG. 1).

FIG. 2 shows the relative expression of MMP1 protein secreted by dermal fibroblasts normalized to beta-acin after being treated with kynurenine and kynurenic acid (reproduced from Poormasjedi-Meibod et al. (2014), FIG. 2).

### DETAILED DESCRIPTION

### I. Definitions

Any terms not directly defined herein shall be understood to have the meanings commonly associated with them as understood within the art of the invention. As employed throughout the specification, the following terms, unless otherwise indicated, shall be understood to have the following meanings.

There are provided herein a number of compounds for use in the treatment of rhytids or wrinkles, or for the reduction in the appearance of rhytids or wrinkles. In the context of the current description, the term rhytids refers to fine lines or folds in the skin, and can colloquially be called lines, wrinkles, or furrows. Although overlap exists, in the context of the current description, the term wrinkles refers to visible folds in the skin, generally deeper and more prominent than rhytids. Both rhytids and wrinkles can be static or dynamic, where dynamic refers to the rhytid(es) or wrinkle(s) that develop from repetition of (and often become visible more apparent, or less apparent, with) underlying muscle movement of the skin; while static refers to the rhytid(es) or wrinkle(s) that develop from gravity, photodamage, or other causes, generally without regard to underlying muscle movement.

In the context of the current description, the term ‘treatment’ may refer to treatment of existing rhytids or wrinkles, or alternately may refer to treatment which occurs before existing rhytids or wrinkles in order to prevent the development or progression them. The compounds described herein may be in isolation, or may be linked to or in combination with tracer compounds, liposomes, carbohydrate carriers, polymeric carriers or other agents or excipients as will be apparent to one of skill in the art. In an alternate embodiment, such compounds may comprise a medicament, wherein such compounds may be present in a pharmacologically effective amount. The compounds may be suitable for administration to a subject in need thereof, by virtue of the fact that the subject may benefit from prophylaxis or treatment of existing rhytids or wrinkles. The compounds may also include tautomers or stereoisomers.

As used herein, KA or KynA may be used as abbreviations for kynurenic acid (CAS #492-27-3) and XA may be used as an abbreviation for xanthurenic acid (CAS #59-00-7). L-kynurenine (CAS #2922-83-0) may be represented herein as L-Kyn and D-kynurenine (CAS #13441-51-5) may be represented herein as D-Kyn. Unless otherwise stated, kynurenine includes L-Kyn, D-Kyn, and a racemic mixture of the two (and where the racemic mixture may be represented as DL-Kyn or DL-Kynurenine, CAS #343-65-7). Stereochemistry of other amino acids may similarly be indicated as D-, L-, or DL- to refer to the racemic mixture thereof. Also contemplated are the salt forms of the compound, for example a salt form of kynurenic acid is the kynurenic acid sodium salt (CAS #2439 Feb. 3).

The term ‘medicament’ as used herein refers to a composition that may be administered to a patient or test subject and is capable of producing an effect in the patient or test subject. The effect may be chemical, biological or physical, and the patient or test subject may be human, or a non-human animal, such as a rodent or transgenic mouse, or a dog, cat, cow, sheep, horse, hamster, guinea pig, rabbit or pig. The medicament may be comprised of the effective chemical entity alone or in combination with a pharmaceutically acceptable excipient.

The term ‘pharmaceutically acceptable excipient’ may include any and all solvents, dispersion media, coatings, antibacterial, antimicrobial or antifungal agents, isotonic and absorption delaying agents, and the like that are physiologically compatible. An excipient may be suitable for intravenous, intraperitoneal, intramuscular, subcutaneous, intrathecal, topical or oral administration. An excipient may include sterile aqueous solutions or dispersions for extemporaneous preparation of sterile injectable solutions or dispersion. Use of such media for preparation of medicaments is known in the art.

The term “Antigen presenting cells (APC)” (also known as accessory cells) refers to cells which display foreign antigen bound to a major histocompatibility complex (MHC) on their cell surface. Subsequently, the APC with antigen bound to MHC may be recognized by T-cells via the T-cell receptors (TCRs). As used herein APC is meant to encompass both non-professional APCs (for example, fibroblasts, thymic epithelial cells, thyroid epithelial cells, glial cells, beta-cells, endothelial cells) and professional APCs (for example, dendritic cells, macrophages, B-cells, and activated epithelial cells). APCs are also meant to encompass both allogenic and syngenic cells.

Compounds or compositions according to some embodiments may be administered in any of a variety of known routes, or formulated for use in administration. Examples of methods that may be suitable for the administration of a compound include orally, intravenous, inhalation, intramuscular, subcutaneous, topical, intraperitoneal, intra-rectal or intra-vaginal suppository, sublingual, and the like. The compounds described herein may be administered as a sterile aqueous solution, or may be administered in a fat-soluble excipient, or in another solution, suspension, patch, tablet or paste format as is appropriate. Other methods known in the art for making formulations are found in, for example, “Remington's Pharmaceutical Sciences”, (19^{th} edition), ed. A. Gennaro, 1995, Mack Publishing Company, Easton, Pa.

The dosage of the compositions or compounds of some embodiments described herein may vary depending on the route of administration (oral, injection, microneedling, topical, or the like) and the form in which the composition or compound is administered (solution, controlled release or the like). Determination of appropriate dosages is within the ability of one of skill in the art. As used herein, an ‘effective amount’, a ‘therapeutically effective amount’, or a ‘pharmacologically effective amount’ of a medicament refers to an amount of a medicament present in such a concentration to result in a therapeutic level of drug delivered over the term that the drug is used. This may be dependent on mode of delivery, time period of the dosage, age, weight, general health, sex and diet of the subject receiving the medicament. As used herein, an “effective amount” means the quantity necessary to render the necessary result. For example, an effective amount of a therapeutic is a level effective to treat, cure, or alleviate the symptoms of a disease for which the therapeutic agent is/are being administered. Methods of determining effective amounts are known in the art.

### II. Biology

As the skin ages it undergoes both biological and physical changes, while more people are living longer and contributing to an increasing number of aged and elderly individuals. A longer life expectancy means more exposure to extrinsic factors that exacerbate the age-related changes that are observed in skin (Venkatesh, 2019). Changes during skin aging include histological changes such as thinning of the skin due to atrophy of the epidermal cell layers and reduction in the components of the extracellular matrix (ECM) in the dermal layer (for example collagen decreases and the collagen becomes fragmented) where the reduction in the ECM and collagen fragmentation is attributable to increased secretion of matrix metalloproteinases by skin fibroblasts with aging (Salminen, 2022). According to Saminen et al., changes in the amount of ECM components “result in many of the clinical features encountered in the aged skin, such as wrinkles and loss of skin elasticity.” (2022, pg. 818)

Venkatesh et al., similarly states that chronological aging is marked by clinical changes such as skin thinning and wrinkles (2019). The skin dermis exhibits decreased collagen synthesis and degraded elastic fibers, and Venkatesh et al. states “[d]egraded elastic fibers and reduced collagen synthesis are responsible for reduced skin elasticity, or skin ptosis, and these alterations contribute to rhytids, or wrinkles.” (2019, pg. 352).

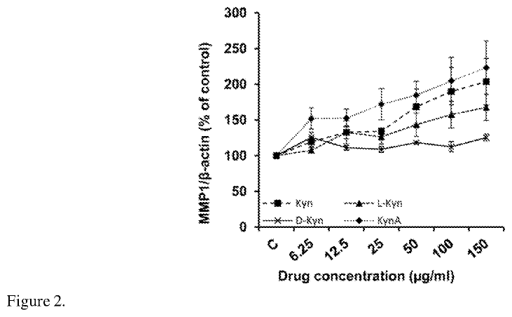

Although it is not necessary to understand the mechanism of an invention and without being bound by any particular theory, the use of kynurenine and other kynurenine pathway metabolites, including kynurenic acid, have previously been described to treat fibroproliferative disorders, namely hypertrophic scarring and keloids (U.S. Pat. No. 9,737,523B2). Fibroproliferative disorders are marked by excessive accumulation of extracellular matrix, and kynurenines (including kynurenic acid) were shown to increase expression MMP-1 and MMP-3 enzymes in dermal fibroblasts ('523 FIGS. 2 and 3, respectively) and downregulate collagen 1 and fibronectin expression by dermal fibroblasts ('523 FIGS. 10 and 16, respectively). Topical application to skin tissue using a rabbit ear hypertrophic scarring model confirmed the dermal fibroblast results, showing that kynurenine decreased collagen and increased MMP-1 relative to controls ('523, FIG. 13). This combination of upregulating known matrix degrading enzymes (i.e., MMP-1 and MMP-3) with concomitant downregulation of collagen 1, as was shown in the '523 patent, effectively reverses hypertrophic scarring in an animal model and effectively reverses hypertrophic keloid scarring in skin of human subjects (BirchBioMed Inc., 2021). However, these data teach away from the use of kynurenines, including kynurenic acid, in treating rhytids and wrinkles. Venkatesh et al. states “[d]egraded elastic fibers and reduced collagen synthesis are responsible for reduced skin elasticity, or skin ptosis, and these alterations contribute to rhytids, or wrinkles.” (2019, pg. 352), while Saminen et al., note that reductions in the amount of ECM components “result in many of the clinical features encountered in the aged skin, such as wrinkles and loss of skin elasticity.” (2022, pg. 818). Upregulating known matrix degrading enzymes (i.e., MMP-1 and MMP-3) with concomitant downregulation of collagen 1 in skin, as was shown in the '523 patent, would accelerate a net loss of skin matrix and would be anticipated to potentiate the formation of rhytids and wrinkles. Put simply, a person skilled in the art would reasonably expect increasing catabolic enzymes (i.e., MMP-1 and MMP-3) and decreasing matrix anabolic proteins (collagen 1) would exacerbate rhytids or wrinkles.

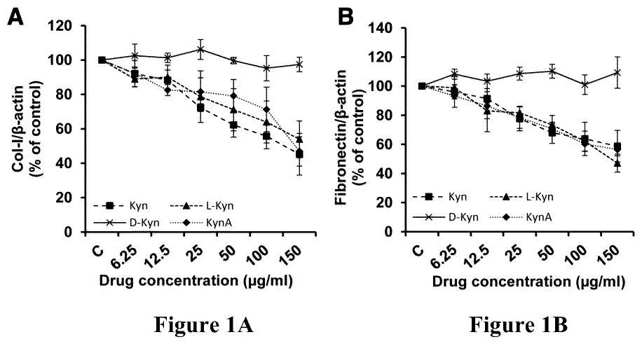

Although it is not necessary to understand the mechanism of an invention and without being bound by any particular theory, Kim et al. reported that aryl hydrocarbon receptor activation is an important contributor to wrinkle formation and that AhR antagonists (inhibition) such as vitamin B12 and folic acid helped reduce UVB-induced wrinkle formation in an animal model (Kim, 2022). Kim's data also showed that the enzyme that produces kynurenic acid (Kyat2) positively correlates with MMP production in skin (Kim, 2022). Important to note, kynurenine and kynurenic acid are known AhR agonists (activators) and in skin will suppress collagen production (illustrated in FIG. 1A) increase MMP production (illustrated in FIG. 2) (Poormasjedi-Meibod, 2014). Together Kim and Poormasjedi-Meibod would thus suggest that kynurenine or kynurenic acid would be expected to be detrimental to trying to resolve, treat, or reduce the appearance of rhytids or wrinkles. Rhytids and wrinkles are due to a lack of ECM and excess MMP, both of these features would be predicted based on Kim and Poormasjedi-Meibod to be exacerbated or worsened by kynurenine, kynurenic acid, or other AhR agonists (AhR activators).

### III. Methods of Compound Use & Formulations

In one embodiment, the present invention contemplates a method to reducing the appearance of rhytids or wrinkles comprising of administering or applying a small molecule compound wherein the compound is selected from the group consisting of DL-kynurenine, L-kynurenine, D-kynurenine, 3-hydroxy-DL-kynurenine, 3-hydroxy-L-kynurenine, 3-hydroxy-D-kynurenine, 5-hydroxy-DL-kynurenine, 5-hydroxy-L-kynurenine, 5-hydroxy-D-kynurenine, N′-formyl-kynurenine, N-Acetyl-3-OH-kynurenine, 4-Chloro-DL-kynurenine, Xanthurenic Acid, and Kynurenic Acid.

In one embodiment, the present invention contemplates a method to treat the appearance of rhytids or wrinkles comprising of administering or applying a small molecule compound wherein the compound is selected from the group consisting of DL-kynurenine, L-kynurenine, D-kynurenine, 3-hydroxy-DL-kynurenine, 3-hydroxy-L-kynurenine, 3-hydroxy-D-kynurenine, 5-hydroxy-DL-kynurenine, 5-hydroxy-L-kynurenine, 5-hydroxy-D-kynurenine, N′-formyl-kynurenine, N-Acetyl-3-OH-kynurenine, 4-Chloro-DL-kynurenine, Xanthurenic Acid, and Kynurenic Acid.

In one embodiment, the present invention contemplates a method to treat rhytids or wrinkles comprising of administering or applying a small molecule compound wherein the compound is selected from the group consisting of DL-kynurenine, L-kynurenine, D-kynurenine, 3-hydroxy-DL-kynurenine, 3-hydroxy-L-kynurenine, 3-hydroxy-D-kynurenine, 5-hydroxy-DL-kynurenine, 5-hydroxy-L-kynurenine, 5-hydroxy-D-kynurenine, N′-formyl-kynurenine, N-Acetyl-3-OH-kynurenine, 4-Chloro-DL-kynurenine, Xanthurenic Acid, and Kynurenic Acid.

In one embodiment, the present invention contemplates a method to reverse rhytids or wrinkles comprising of administering or applying a small molecule compound wherein the compound is selected from the group consisting of DL-kynurenine, L-kynurenine, D-kynurenine, 3-hydroxy-DL-kynurenine, 3-hydroxy-L-kynurenine, 3-hydroxy-D-kynurenine, 5-hydroxy-DL-kynurenine, 5-hydroxy-L-kynurenine, 5-hydroxy-D-kynurenine, N′-formyl-kynurenine, N-Acetyl-3-OH-kynurenine, 4-Chloro-DL-kynurenine, Xanthurenic Acid, and Kynurenic Acid.

In one embodiment, the present invention contemplates a method to prevent rhytids or wrinkles comprising of administering or applying a small molecule compound wherein the compound is selected from the group consisting of DL-kynurenine, L-kynurenine, D-kynurenine, 3-hydroxy-DL-kynurenine, 3-hydroxy-L-kynurenine, 3-hydroxy-D-kynurenine, 5-hydroxy-DL-kynurenine, 5-hydroxy-L-kynurenine, 5-hydroxy-D-kynurenine, N′-formyl-kynurenine, N-Acetyl-3-OH-kynurenine, 4-Chloro-DL-kynurenine, Xanthurenic Acid, and Kynurenic Acid.

In one embodiment, the present invention contemplates a method to conceal rhytids or wrinkles comprising of administering or applying a small molecule compound wherein the compound is selected from the group consisting of DL-kynurenine, L-kynurenine, D-kynurenine, 3-hydroxy-DL-kynurenine, 3-hydroxy-L-kynurenine, 3-hydroxy-D-kynurenine, 5-hydroxy-DL-kynurenine, 5-hydroxy-L-kynurenine, 5-hydroxy-D-kynurenine, N′-formyl-kynurenine, N-Acetyl-3-OH-kynurenine, 4-Chloro-DL-kynurenine, Xanthurenic Acid, and Kynurenic Acid.

In one embodiment the, the present invention contemplates a method to prevent rhytids or wrinkles comprising of administering or applying a small molecule compound wherein the compound is selected from the group consisting of DL-kynurenine, L-kynurenine, D-kynurenine, 3-hydroxy-DL-kynurenine, 3-hydroxy-L-kynurenine, 3-hydroxy-D-kynurenine, 5-hydroxy-DL-kynurenine, 5-hydroxy-L-kynurenine, 5-hydroxy-D-kynurenine, N′-formyl-kynurenine, N-Acetyl-3-OH-kynurenine, 4-Chloro-DL-kynurenine, Xanthurenic Acid, and Kynurenic Acid.

In one embodiment, the present invention contemplates a method to treat, reverse, conceal, or reduce the appearance of rhytids or wrinkles comprising of administering or applying a small molecule compound wherein the compound is selected from the group consisting of DL-kynurenine, L-kynurenine, D-kynurenine, 3-hydroxy-DL-kynurenine, 3-hydroxy-L-kynurenine, 3-hydroxy-D-kynurenine, 5-hydroxy-DL-kynurenine, 5-hydroxy-L-kynurenine, 5-hydroxy-D-kynurenine, N′-formyl-kynurenine, N-Acetyl-3-OH-kynurenine, 4-Chloro-DL-kynurenine, Xanthurenic Acid, and Kynurenic Acid.

In one embodiment the, the present invention contemplates a small molecule compound wherein the compound is selected from the group consisting of DL-kynurenine, L-kynurenine, D-kynurenine, 3-hydroxy-DL-kynurenine, 3-hydroxy-L-kynurenine, 3-hydroxy-D-kynurenine, 5-hydroxy-DL-kynurenine, 5-hydroxy-L-kynurenine, 5-hydroxy-D-kynurenine, N′-formyl-kynurenine, N-Acetyl-3-OH-kynurenine, 4-Chloro-DL-kynurenine, Xanthurenic Acid, and Kynurenic Acid for the treatment of rhytids or wrinkles.

In one embodiment, the present invention contemplates a small molecule compound wherein the compound is selected from the group consisting of DL-kynurenine, L-kynurenine, D-kynurenine, 3-hydroxy-DL-kynurenine, 3-hydroxy-L-kynurenine, 3-hydroxy-D-kynurenine, 5-hydroxy-DL-kynurenine, 5-hydroxy-L-kynurenine, 5-hydroxy-D-kynurenine, N′-formyl-kynurenine, N-Acetyl-3-OH-kynurenine, 4-Chloro-DL-kynurenine, Xanthurenic Acid, and Kynurenic Acid for the reduction in the appearance of rhytids or wrinkles.

In one embodiment, the present invention contemplates a compound selected from the group consisting of DL-kynurenine, L-kynurenine, D-kynurenine, 3-hydroxy-DL-kynurenine, 3-hydroxy-L-kynurenine, 3-hydroxy-D-kynurenine, 5-hydroxy-DL-kynurenine, 5-hydroxy-L-kynurenine, 5-hydroxy-D-kynurenine, N′-formyl-kynurenine, N-Acetyl-3-OH-kynurenine, 4-Chloro-DL-kynurenine, Xanthurenic Acid, and Kynurenic Acid, for use in the treatment of rhytids or wrinkles. In one embodiment, kynurenic acid for use in the treatment of rhytids or wrinkles. In one embodiment, the compound is kynurenic acid for use in the treatment of rhytids or wrinkles. In one embodiment, kynurenic acid is a component of a composition formulated for topical administration for use in the treatment of rhytids or wrinkles. In one embodiment, kynurenic acid is a component of a composition formulated for topical administration for use in the concealment of rhytids or wrinkles. In one embodiment, kynurenic acid is a component of a composition formulated for topical administration for use as a cosmetic treatment of rhytids or wrinkles. In one embodiment, kynurenic acid is a component of a composition formulated for topical administration for use as a cosmetic product for reducing the appearance of rhytids or wrinkles. In one embodiment, the compound is kynurenine for use in the treatment of rhytids or wrinkles. In one embodiment, the compound is kynurenine for use in the treatment of rhytids or wrinkles.

In one embodiment, the compound is a component of a composition formulated for injection and is administered by injection. In one embodiment the injection is administered as a subcutaneous, intradermal, or an intramuscular injection. In another embodiment, the compound is a component of a composition formulated for platelet-rich plasma (PRP) therapy or platelet-rich fibrin (PRF) therapy. In yet another embodiment, the compound is a component of a composition formulated for microneedling with PRP or PRF. In a further embodiment, the compound is administered or applied in combination with platelet-rich plasma (PRP) therapy or platelet-rich fibrin (PRF) therapy. The PRP or the PRF may be co-administered with the compound. The co-administration may be simultaneous. The co-administration may be consecutive. The PRP or PRF may be administered before the compound. The compound may be administered before the PRP or PRF.

In another embodiment, an antigen presenting cell (APC) is presented to a mammal and the compound is administered to the mammal. The APC may be allogenic. The APC may be syngenic. The APC may be a non-professional APC. The APC may be a professional APC. The APC and the compound may be co-administered. The co-administration may be simultaneous. The co-administration may be consecutive. The APC may be administered before the compound. The compound may be administered before the APC. The APC may be administered intraperitoneally. The compound may be administered orally. The APC may be selected from one or more of the following: fibroblasts, thymic epithelial cells, thyroid epithelial cells, glial cells, beta-cells, and endothelial cells. The APCs may be fibroblast cells. The mammal may be a human. The mammal may be a human with rhytids and/or wrinkles. The mammal may be a human with rhytids and/or wrinkles, the APC may be a fibroblast. The method may further include periodic compound administration following the initial administration of APC and the compound. The compound may be selected from one of more of DL-kynurenine, L-kynurenine, D-kynurenine, and 3-hydroxy-DL-kynurenine. The kynurenine may be selected from one of more of DL-kynurenine, L-kynurenine, D-kynurenine, 3-hydroxy-DL-kynurenine, 3-hydroxy-L-kynurenine, 3-hydroxy-D-kynurenine, 5-hydroxy-DL-kynurenine, 5-hydroxy-L-kynurenine, 5-hydroxy-D-kynurenine, N′-formyl-kynurenine, N-Acetyl-3-OH-kynurenine, 4-Chloro-DL-kynurenine, Kynurenine, butyl ester, and Kynurenic Acid. The kynurenine may be an isomer of kynurenine or an analog thereof.

In one embodiment, the compound is a component of a composition formulated for oral delivery. In one embodiment the compound is an ingredient in a product intended for oral consumption.

In one embodiment, the compound is a component of a composition formulated for topical delivery. In one embodiment the compound is a component of a composition formulated for topical application. In one embodiment the compound is an ingredient in a lotion, cream, gel, solution, or suspension for topical use.

In some embodiments, the application, administration, or use of the compound is contemplated as one to five times per day. In some embodiments, the application, administration, or use of the compound is contemplated as once, twice, or thrice daily. In some embodiments, the application, administration, or use is contemplated as topical.

In one embodiment, the compound is selected from the group consisting of DL-kynurenine, L-kynurenine, D-kynurenine, 3-hydroxy-DL-kynurenine, 3-hydroxy-L-kynurenine, 3-hydroxy-D-kynurenine, 5-hydroxy-DL-kynurenine, 5-hydroxy-L-kynurenine, 5-hydroxy-D-kynurenine, N′-formyl-kynurenine, N-Acetyl-3-OH-kynurenine, 4-Chloro-DL-kynurenine, Xanthurenic Acid, and Kynurenic Acid, and is a component of a composition formulated for topical use or topical application.

In one embodiment, the compound is selected from the group consisting of DL-kynurenine, L-kynurenine, D-kynurenine, 3-hydroxy-DL-kynurenine, 3-hydroxy-L-kynurenine, 3-hydroxy-D-kynurenine, 5-hydroxy-DL-kynurenine, 5-hydroxy-L-kynurenine, 5-hydroxy-D-kynurenine, N′-formyl-kynurenine, N-Acetyl-3-OH-kynurenine, 4-Chloro-DL-kynurenine, Xanthurenic Acid, and Kynurenic Acid, and is a component of a composition formulated as a cream, gel, lotion, foam, suspension, or ointment.

In one embodiment, the compound is selected from the group consisting of DL-kynurenine, L-kynurenine, D-kynurenine, 3-hydroxy-DL-kynurenine, 3-hydroxy-L-kynurenine, 3-hydroxy-D-kynurenine, 5-hydroxy-DL-kynurenine, 5-hydroxy-L-kynurenine, 5-hydroxy-D-kynurenine, N′-formyl-kynurenine, N-Acetyl-3-OH-kynurenine, 4-Chloro-DL-kynurenine, Xanthurenic Acid, and Kynurenic Acid, and is formulated with an additional one or more pharmaceutical composition(s), wherein said pharmaceutical compositions include a retinoid or retinoid-like compound (e.g., tretoin, adapalene, tazorotene).

In one embodiment, the compound is selected from the group consisting of DL-kynurenine, L-kynurenine, D-kynurenine, 3-hydroxy-DL-kynurenine, 3-hydroxy-L-kynurenine, 3-hydroxy-D-kynurenine, 5-hydroxy-DL-kynurenine, 5-hydroxy-L-kynurenine, 5-hydroxy-D-kynurenine, N′-formyl-kynurenine, N-Acetyl-3-OH-kynurenine, 4-Chloro-DL-kynurenine, Xanthurenic Acid, and Kynurenic Acid, and is formulated with an additional one or more non-prescription drugs, wherein said non-prescription drug(s) is covered by an FDA OTC monograph(s). Non-limiting examples of said non-prescription drugs include salicylic acid, benzoyl peroxide, allantoin, cocoa butter, dimethicone, glycerin, petrolatum, live yeast cell derivative (LYCD), zinc stearate, zinc acetate, zinc carbonate, zinc oxide, mineral oil, resorcinol, Peruvian balsalm, shark liver oil, and tannic acid.

In one embodiment, the compound is kynurenic acid and is between 0.1% and 1% by weight of a composition for topical use or topical application. In one embodiment, the compound is kynurenic acid and is 0.25% to 0.5% by weight of a composition for topical use or topical application. In one embodiment, the compound is kynurenic acid at 0.5% by weight of a composition for topical use or topical application. In one embodiment, the compound is kynurenic acid and is 0.5% of a lotion. In one embodiment, the compound is kynurenic acid and is 0.5% of a cream. In one embodiment, the compound is kynurenic acid and is 0.1% to 2% by weight of a composition for topical application or topical use for use in reducing the appearance of rhytids or wrinkles. In one embodiment, the compound is kynurenic acid and is 0.1% to 2% by weight of a composition for use as a cosmetic product to reduce the appearance of rhytids or wrinkles.

In one embodiment, the present invention contemplates the use of kynurenic acid in the manufacture of a cosmetic or drug product to reduce the appearance of rhytids or wrinkles. In one embodiment, the present invention contemplates a topical formulation containing a trace to 2% by weight of kynurenine, kynurenic acid, or xanthurenic acid; and the use of said formulation for reducing the appearance of rhytids or wrinkles. In one embodiment, the present invention contemplates a topical formulation containing trace to 2% by weight of kynurenine, kynurenic acid, or xanthurenic acid; and said formulation for use in reducing the appearance of rhytids or wrinkles. In one embodiment, the present invention contemplates a topical formulation containing 0.1 to 0.75% by weight of kynurenine, kynurenic acid, or xanthurenic acid; and said formulation for use in reducing the appearance of rhytids or wrinkles. In one embodiment, the present invention contemplates a topical formulation containing 0.25 to 0.5% by weight of kynurenine, kynurenic acid, or xanthurenic acid; and said formulation for use in reducing the appearance of rhytids or wrinkles. In one embodiment, the present invention contemplates a topical formulation containing 0.1 to 0.75% by weight of kynurenic acid; and said formulation for use in reducing the appearance of rhytids or wrinkles.

### IV. Examples

### A. Formulations for Topical Use

I. A topical cream was made as follows, kynurenic acid was solubilized in a phosphate buffered solution of 1 M NaOH and the pH was then adjusted to 5.5 at room temperature, this KynA solution was then added with steady mixing to a dermatological compounding base (Glaxal Base™, WellSpring, Ont., Canada) and adjusted to a pH of 6 before packaging in poly bottles. Topical cream was made at 0.15%, 0.25%, 0.4%, and 0.5% kynurenic acid by weight (Papp et al., 2018).

II. Other compounding creams are known in the art, and an example is the VersaPro Cream Base (Product #2529, MEDISCA Pharmaceutique Inc., Richmond, BC, Canada). Using the VersaPro Cream Based, dry kynurenic acid powder to make a 0.5% final weight was manually mixed into the cream using a mortar and pestle.

III. Moisturizing Cream with Kynurenic Acid

A 0.5% kynurenic acid by weight moisturizing cream was made by combining water, petrolatum, cetearyl alcohol, light mineral oil, ceteareth-20, TroyCare™ EPP37, sodium dihydrogen phosphate dihydrate, kynurenic acid, sodium hydroxide, hydrochloric acid, sodium chloride, and disodium hydrogen phosphate dihydrate. A 0.25% by weight kynurenic acid moisturizing cream was made by combining the same ingredients (water, petrolatum, cetearyl alcohol, light mineral oil, ceteareth-20, TroyCare™ EPP37, sodium dihydrogen phosphate dihydrate, kynurenic acid, sodium hydroxide, hydrochloric acid, sodium chloride, and disodium hydrogen phosphate dihydrate), wherein the amount of kynurenic acid was reduced to 0.25% of the final weight of the product.

### B. Use of Topical Kynurenic Acid (0.5%, by Weight) for Reducing Appearance of Rhytids and Wrinkles (Female).

A female in her 60's with typical rhytids and wrinkles on the face applied a topical cream containing 0.5% kynurenic acid by weight twice daily at approximately 3 mg of kynurenic acid per application and a rate of approximately 60 micrograms per square cm. Initial dermatological effects can be followed by visual inspection of the site. Over an approximately two-month period the subject reported a perceptible and noticeable reduction in their appearance and visibility of her rhytids and wrinkles. The subject also reported that others commented on the reduced appearance of her rhytids and wrinkles after she began application of the kynurenic acid cream.

### C. Use of Topical Kynurenic Acid (0.5% by Weight) for Reducing the Appearance of Rhytids and Wrinkles (Male).

A male of approx. 70 years of age with rhytids and wrinkles on the face self-reported that the applied a topical cream containing 0.5% kynurenic acid by weight once a day for a 4-month period at an application of approximately 3 mg of kynurenic acid per application and a rate of approximately 80 micrograms of kynurenic acid per square cm per application. Initial dermatological effects can be followed by visual inspection of the site. The user reported that after a 2-month period, his rhytids and wrinkles became less visible and less noticeable and appeared to be resolving. The subject also reported that others had also remarked that the user's rhytids and wrinkles were less visible and less noticeable.

D. Use of Topical Kynurenine for Hypertrophic Scarring, which is Envisioned for Use in Treating Rhytids and Wrinkles.

Topical kynurenine cream at 0.05% by weight has been described in literature for use in treating hypertrophic scarring in vivo (Li et al., 2014; Poormasjedi-Meibod et al., 2014), and methods of cream manufacture and topical application are incorporated by reference. This topical kynurenine cream at 0.05% by weight can be applied topically for treating rhytids and wrinkles.

### E. Clinical Classification of Rhytids and Wrinkles

Classification schemes are described in the art for rhytids and wrinkles, and examples of these include: Glogau's classification scheme of Mild (few wrinkles, requires little or no make-up for coverage), Moderate (early wrinkling, sallow complexion, requires little makeup), Advanced (persistent wrinkling, skin discolouration with broken blood vessels and actinic keratoses, often wears make-up), and Severe (severe wrinkling and furrows, actinic keratoses, often wears make-up but it may not hide the ageing changes). Another example is the Fitzpatrick classification of facial lines, typically used to refer to areas around the mouth and eyes, of Class I (fine wrinkles), Class II (fine-to-moderately deep wrinkles and moderate number of lines), and Class III (fine-to-deep wrinkles, numerous lines, and possibly redundant folds). An additional example is the modified Fitzpatrick classification on a scale of 0 (no wrinkle, no visible wrinkle, continuous skin line) to 3 (deep wrinkle, deep and furrow wrinkle, greater than 3 mm wrinkle depth).

### REFERENCES

Chaudhary, M., Khan, A., & Gupta, M. (2020). Skin ageing: Pathophysiology and current market treatment approaches. Current Aging Science, 13(1), 22-30.
Eldin, R. M. S., Nassar, A. A., & Awad, H. Updated Management of Forehead Wrinkles: An Overview. European Journal of Molecular & Clinical Medicine, 8(03), 2021.
Ferreira, M. S., Magalhães, M. C., Sousa-Lobo, J. M., & Almeida, I. F. (2020). Trending anti-aging peptides. Cosmetics, 7(4), 91.
Kim, D. J., Iwasaki, A., Chien, A. L., & Kang, S. (2022). UVB-mediated DNA damage induces matrix metalloproteinases to promote photoaging in an AhR- and SP1-dependent manner. JCI insight, 7(9).
Li, Y., Kilani, R. T., Rahmani-Neishaboor, E., Jalili, R. B., & Ghahary, A. (2014). Kynurenine increases matrix metalloproteinase-1 and -3 expression in cultured dermal fibroblasts and improves scarring in vivo. Journal of Investigative Dermatology, 134(3), 643-650.
Papp, A., Hartwell, R., Evans, M., & Ghahary, A. (2018). The Safety and Tolerability of Topically Delivered Kynurenic Acid in Humans. A Phase 1 Randomized Double-Blind Clinical Trial. J Pharm Sci, 107(6), 1572-1576. doi: 10.1016/j.xphs.2018.01.023.
Poormasjedi-Meibod, M. S., Hartwell, R., Taghi Kilani, R., & Ghahary, A. (2014). Anti-scarring properties of different tryptophan derivatives. PloS one, 9(3), e91955.
Salminen, A., Kaarniranta, K., & Kauppinen, A. (2022). Photoaging: UV radiation-induced inflammation and immunosuppression accelerate the aging process in the skin. Inflammation Research, 1-15.
Tran, H. M., Yang, C. Y., Wu, T. H., & Yen, F. L. (2022). Liposomes Encapsulating Morin: Investigation of Physicochemical Properties, Dermal Absorption Improvement and Anti-Aging Activity in PM-Induced Keratinocytes. Antioxidants, 11(6), 1183.
Venkatesh, S., Maymone, M. B., & Vashi, N. A. (2019). Aging in skin of color. Clinics in dermatology, 37(4), 351-357.

All publications, patent applications, patents, and other references mentioned herein are expressly incorporated by reference in their entirety, to the same extent as if each were incorporated by reference individually. In case of conflict, the present specification, including definitions, will control.

This invention has been described by reference to certain preferred embodiments, however, it should be understood that it may be embodied in other specific forms or variations thereof without departing from its special or essential characteristics. The embodiments described above are, therefore, considered to be illustrative in all respects and not restrictive, the scope of the invention being indicated by the appended claims rather than by the foregoing description.

1-28. (canceled)

29. A method to reduce the appearance of rhytids or wrinkles comprising of administering or applying a small molecule compound wherein the compound is selected from the group consisting of DL-kynurenine, L-kynurenine, D-kynurenine, 3-hydroxy-DL-kynurenine, 3-hydroxy-L-kynurenine, 3-hydroxy-D-kynurenine, 5-hydroxy-DL-kynurenine, 5-hydroxy-L-kynurenine, 5-hydroxy-D-kynurenine, N′-formyl-kynurenine, N-Acetyl-3-OH-kynurenine, 4-Chloro-DL-kynurenine, Xanthurenic Acid, and Kynurenic Acid.

30. A method of claim 29, wherein the small molecule compound is administered or applied as a component of a composition formulated for topical use.

31. A method of claim 30, wherein the composition is formulated as a cream.

32. A method of claim 30, wherein the compound is kynurenic acid and the composition is formulated as 0.5% kynurenic acid by weight.

33. A method of claim 30, wherein the compound is kynurenine and the composition is formulated as 0.05% kynurenine by weight.

34. A method of claim 30, wherein said administering or applying is once or twice daily.

35. A method of claim 30, wherein said administering or applying is topically to skin with rhytids or wrinkles.

36. A method of claim 29, wherein the small molecule compound is administered as a component of a composition formulated for use by injection.

37. A method of claim 29, wherein the small molecule compound is a component of a composition formulated for platelet-rich plasma (PRP) therapy or platelet-rich fibrin (PRF) therapy.

38. A method of claim 30, wherein said administering or applying is topically to skin with rhytids or wrinkles in combination with administration or application of platelet-rich plasma (PRP) therapy or platelet-rich fibrin (PRF) therapy.

39. A method of claim 30, wherein said administering or applying is topically to skin with rhytids or wrinkles in combination with administration or application of an antigen presenting cell (APC).

40. A method to treat rhytids or wrinkles comprising of administering or applying a small molecule compound wherein the compound is selected from the group consisting of DL-kynurenine, L-kynurenine, D-kynurenine, 3-hydroxy-DL-kynurenine, 3-hydroxy-L-kynurenine, 3-hydroxy-D-kynurenine, 5-hydroxy-DL-kynurenine, 5-hydroxy-L-kynurenine, 5-hydroxy-D-kynurenine, N′-formyl-kynurenine, N-Acetyl-3-OH-kynurenine, 4-Chloro-DL-kynurenine, Xanthurenic Acid, and Kynurenic Acid.

41. A method of claim 40, wherein the small molecule compound is administered or applied as a component of a composition formulated for topical use.

42. A method of claim 41, wherein the composition is formulated as a cream.

43. A method of claim 40, wherein the small molecule compound is administered as a component of a composition formulated for use by injection.

44. A method of claim 40, wherein the small molecule compound is a component of a composition formulated for platelet-rich plasma (PRP) therapy or platelet-rich fibrin (PRF) therapy.

45. A method of claim 41, wherein said administering or applying is topically to skin with rhytids or wrinkles in combination with administration or application of platelet-rich plasma (PRP) therapy or platelet-rich fibrin (PRF) therapy.

46. A method of claim 41, wherein said administering or applying is topically to skin with rhytids or wrinkles in combination with administration or application of an antigen presenting cell (APC).

47. A method of claim 41, wherein the compound is kynurenic acid and the composition is formulated as 0.5% kynurenic acid by weight.

48. A method of claim 41, wherein the compound is kynurenine and the composition is formulated as 0.05% kynurenine by weight.

49. A method of claim 41, wherein said administering or applying is once or twice daily.
